(19) United States
(12) Patent Application Publication
(10) Pub. No.: US 2024/0249641 A1
(43) Pub. Date: Jul. 25, 2024

(54) HIGH-SPEED READING TEST METHOD USING EYE TRACKING

(71) Applicants: Gyu-Jin Park, Seongnam-si (KR); Se-Ho Park, Seongnam-si (KR)

(72) Inventors: Gyu-Jin Park, Seongnam-si (KR); Se-Ho Park, Seongnam-si (KR)

(21) Appl. No.: 18/543,835

(22) Filed: Dec. 18, 2023

(30) Foreign Application Priority Data
Jan. 20, 2023 (KR) 1020230008482

(51) Int. Cl. G09B 17/00; A61B 5/00; A61B 5/16; G09B 17/04

(52) U.S. Cl. CPC G09B 17/003; A61B 5/163; A61B 5/742; G09B 17/04

(57) ABSTRACT

The present invention relates to a high-speed reading test method using eye tracking, which tracks the gaze of a subject while the subject reads a given text within a predetermined period of time, extracts a gaze point on the text and the movement of the gaze point, and preliminarily distinguishes whether the subject has fully read the text within the time limit using the extracted gaze point, thereby determining the subject's reading ability deterioration. The high-speed reading test method using eye tracking, which displays diagnostic texts through a display device, and acquires gaze results of a subject who has read the diagnostic texts by an eye tracking device to assess reading ability, includes the steps of: (a) outputting diagnostic texts to be tested, and limiting to read only during a pre-determined test duration; (b) tracking the gaze of the subject to acquire gaze results within a text area, wherein the gaze results includes gaze points displayed within the diagnostic texts, and links connecting the gaze points; (c) determining, based on the gaze results of the subject, whether the text has been fully read; and (d) classifying the subject's reading ability based on whether the text is fully read or not within the limited time. The high-speed reading test method can standardize test times uniformly by requiring a user to read only a given text within a given period of time to test, thereby efficiently reducing the time for mass reading ability tests and quickly determining deterioration in reading ability.

### BACKGROUND OF THE INVENTION

### Field of the Invention

The present invention relates to a high-speed reading test method using eye tracking, which tracks the gaze or the fixation of a subject while the subject reads a given text within a predetermined period of time, extracts a gaze point on the text and the movement of the gaze point, and preliminarily distinguishes whether the subject has fully read the text within the time limit using gaze point, thereby the extracted determining the subject's reading ability deterioration.

### Background Art

In general, literacy is the ability to read and understand written materials, and it is divided into four developmental stages: a first stage of phonemic awareness; a second stage of vocabulary; a third stage of fluency (the ability to read words and sentences quickly and accurately); and a fourth stage of comprehension.

However, in the United States, 52% of the reading population has reading disorders. Such reading disorders must be detected and treated at the early stage. That is, since reading disorders require changing reading habits for treatment, early treatment is highly effective. For this, a method for testing reading ability must first be developed.

To test reading ability, eye tracking techniques have been proposed (Patent Document 1). Eye tracking is a scientific tool that can easily test the level of human reading ability. The eye tracking allows for measuring the time taken to read a text, vocabulary, and intensive reading ability.

Specifically, conventional arts utilize all eye tracking record data, such as a fixation duration of human eyes, jump amplitude, whether to do being-watching or not, gaze duration (average fixation duration), jump range (average jump distance), type and distribution of jumps (normal/line change/retrogression/exit, etc.), and a retrogression rate of eyes, to assess a user's ability. For example, to measure reading speed, the conventional art track till a student reads a text from beginning to end, and calculate the average reading speed by dividing the total time taken by the number of words or characters.

However, the conventional arts complicatedly analyze a subject's reading ability by extracting too many variables through eye tracking. Especially, since the conventional arts require the subject to read the entire given text, the time taken to read the text varies greatly among subjects. Therefore, the conventional arts have a problem in that it is difficult to standardize since the test time is varied according to subjects. Furthermore, subject reads slowly, the test time is significantly prolonged, so the conventional arts require lots of test time.

In particular, the conventional arts are not suitable for mass reading ability tests targeting the entire population. That is, the conventional arts do not meet the requirements of real-world situations where large-scale eye tracking is needed and, when analyzing the results of eye tracking later, are complicated since extracting too many analysis items.

### PATENT LITERATURE

### Patent Documents

Patent Document 1: Korean Patent No. 10-2262890 (published on Jun. 9, 2021)

### SUMMARY OF THE INVENTION

Accordingly, the present invention has been made to solve the above-mentioned problems occurring in the prior arts, and it is an objective of the present invention to provide a high-speed reading test method using eye tracking, which tracks the gaze of a subject while the subject reads a given text within a predetermined period of time, extracts a gaze point on the text and the movement of the gaze point, and preliminarily distinguishes whether the subject has fully read the text within the time limit using the extracted gaze point, thereby determining the subject's reading ability deterioration.

It is another objective of the present invention to provide a high-speed reading test method using eye tracking, which, based on whether the text is fully read or not, in cases of incomplete reading, distinguishes the lack of vocabulary and fluency by excessive size and insufficient movement of gaze points, and in cases of complete reading, distinguishes reading ability deterioration according to patterns of eye movements.

To accomplish the above object, according to the present invention, there is provided a high-speed reading test method using eye tracking, which displays diagnostic texts through a display device, and acquires gaze results of a subject who has read the diagnostic texts by an eye tracking device to assess reading ability, including the steps of: (a) outputting diagnostic texts to be tested, and limiting to read only during a pre-determined test duration; (b) tracking the gaze of the subject to acquire gaze results within a text area, wherein the gaze results includes gaze points displayed within the diagnostic texts, and links connecting the gaze points; (c) determining, based on the gaze results of the subject, whether the text has been fully read; and (d) classifying the subject's reading ability based on whether the text is fully read or not within the limited time.

Moreover, in the step (d), when the text has not been not fully read within the limited time, a type 1 reading ability deterioration is determined.

Furthermore, in the step (d), when the text has not been fully read within the limited time, a lack of vocabulary is determined by excessive gaze points larger than a pre-determined threshold size, or a lack of fluency is determined by using insufficient movement, where a series of intervals between the than a pre-determined minimum gaze points each move less threshold interval.

Additionally, in the step (d), when the text has been fully read within the limited time, a type 2 reading ability deterioration is determined based on whether there are excessive leaps or saccades in the eye leaps at line changes of the diagnostic text.

In addition, in the step (d), when the text has been fully read within the limited time, if the number of characters in a line change link exceeds a pre-determined threshold character count, it is determined that excessive leaps at line changes (hereinafter, line change excessive leaps) have occurred, and if the number or proportion of line change excessive leaps exceeds a pre-determined number or proportion, it is determined as the type 2 reading ability deterioration.

Additionally, the present invention relates to a computer-readable recording medium in which a program for executing a high-speed reading test method using eye tracking is recorded.

As described above, the high-speed reading test method using eye tracking according to the present invention can standardize test times uniformly by requiring a user to read only a given text within a given period of time to test, thereby efficiently reducing the time for mass reading ability tests and quickly determining deterioration in reading ability.

Furthermore, the high-speed reading test method using eye tracking according to the present invention can preliminarily distinguish whether the text is completely read, and then, based on the result, distinguish reading ability in detail, thereby testing deterioration in reading ability simply, quickly, and accurately.

### BRIEF DESCRIPTION OF THE DRAWINGS

FIG. 1 is a configuration diagram of the whole system for realizing the present invention.

FIG. 2 is a flowchart depicting a high-speed reading test method using eye tracking according to an embodiment of the present invention.

FIG. 3 is an exemplary diagram of a diagnostic text according to an embodiment of the present invention.

FIG. 4 is an exemplary diagram of gaze results based on the eye tracking of an embodiment of the present invention.

FIG. 5 is an exemplary diagram of a Type 1 reading ability deterioration according to an embodiment of the present invention.

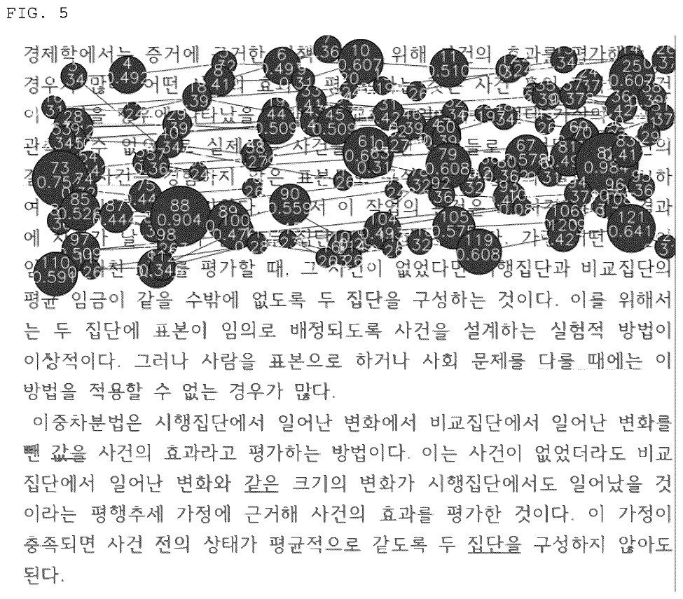

FIG. 6 is an exemplary diagram of a Type 2 reading ability deterioration according to an embodiment of the present invention.

FIG. 7 is an exemplary diagram of the constant-speed type according to an embodiment of the present invention.

### DETAILED DESCRIPTION OF THE PREFERRED EMBODIMENT

Hereinafter, specific contents for realizing the present invention will be described with reference to the drawings.

Moreover, in describing the present invention, the same parts are denoted by the same reference numerals, and repeated descriptions will be omitted.

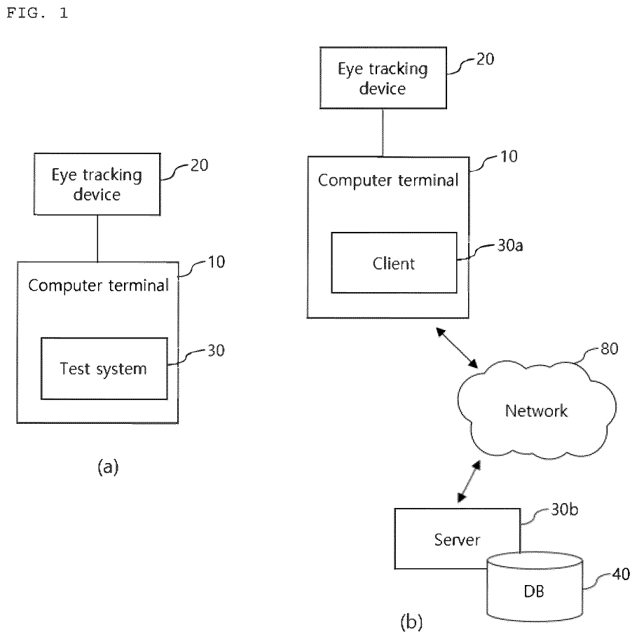

First, the configuration of the whole system for realizing the present invention is described with reference to FIG. 1.

As illustrated in FIG. 1(a), a high-speed reading test method using eye tracking according to the present invention can be implemented as a program system 30 on a computer terminal 10, which determines the reading ability of a subject by extracting a gaze area through eye tracking of an eye tracking device 20.

That is, the test method can be implemented by a program system 30 on a computer terminal 10, such as a PC, a smartphone, a tablet PC, etc. Particularly, the test method is configured as a program system 30 and can be installed and executed on the computer terminal 10. The test method, by using hardware or software resources of the computer terminal 10, provides services of extracting a gaze area through eye tracking to and determining a subject's reading ability.

Meanwhile, a diagnostic text is displayed on a display device, which can be a separate display device or a display device of the computer terminal 10.

Furthermore, as another embodiment, as illustrated in FIG. 1(b), the method can be executed by being configured as a server-client system 30 including a client 30a and a server 30b on the computer terminal 10. In this case, the client 30a acquires the eye tracking results through an eye-tracking device 20 connected to the computer terminal 10. Additionally, the display device may be a separate device or the display device of the computer terminal.

Meanwhile, the client 30a and the server 30b can be realized according to the conventional configuration methods of clients and servers. That is, the functions of the entire system can be allocated according to the performance of the client or the communication t with the server. Various forms of allocation can be realized according to the configuration method of the server-client.

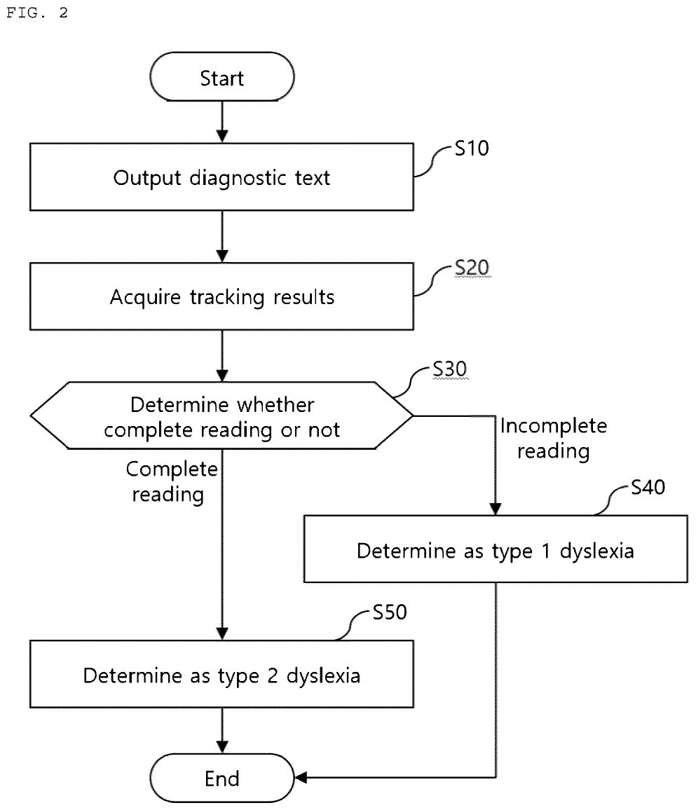

Next, referring to FIG. 2, the high-speed reading test method 30 using eye tracking according to an embodiment of the present invention will be described.

As illustrated in FIG. 2, the high-speed reading test method using eye tracking according to an embodiment of the present invention includes: a step (S10) of outputting a diagnostic text to be tested; a step (S20) of tracking a subject's eyes to acquire the gaze results within a text area; a step (S30) of determining whether the text has been fully read from the gaze results; and a step (S40) of, if not fully read, determining a Type 1 reading ability deterioration. Additionally, if fully read, the method comprises a step (S40) of diagnosing a Type 2 reading ability deterioration based on excessive leaps (S40).

First, the diagnostic text to be tested is displayed on a screen, especially displayed only within a predetermined time (or limit time) (S10). That is, there is a limit in the time for viewing the text (or test time). In other words, the test time is set within a predetermined limit time.

Furthermore, the diagnostic text is randomly selected from pre-stored diagnostic texts and displayed on the screen. That is, the predetermined multiple diagnostic texts are stored in a database, which is referred to as a text database.

Preferably, the diagnostic texts can be grouped and classified according to the subject's group. Text groups can be classified by the subject's age, education level, profession, etc. For example, depending on the age of the subject, the text groups can be classified into elementary students, middle school students, high school students, adults, etc., or into age groups like teens, 20s to 30s, 40s to 50s, 60s, and above. In other words, since each group has different vocabulary levels and reading speeds, text amounts and contents suitable for the respective subject groups are prepared in advance.

Additionally, the test time (or examination time) can be varied according to the group of the subject.

In this instance, the diagnostic texts can be selected or extracted by experts, or selected based on other reading ability-related data of the corresponding group. For example, for high school students, texts can be selected or time limits can be set based on the reading speed necessary and sufficient for solving problems of Korean language test in the national college entrance examination. That is, the word count and vocabulary level of the diagnostic texts can be determined considering the level of texts in past national college entrance examination questions or equivalent. Furthermore, the time limit for the corresponding group (high school student group) can be set based on the reading speed of texts in national college entrance examination questions or the time taken to read the texts in full.

Additionally, adults (or the general public) are considered to be on a par with high school students, so for the high school and adult groups, diagnostic texts are selected or time limits are set considering the vocabulary level and reading time of the national college entrance examination texts. For elementary and middle school students, texts and time limits can be adjusted downwards based on the criteria for high school students and adults. Alternatively, texts and time limits can be determined by reflecting texts of other examinations such as the joint academic assessment for middle school students.

In addition, subjects are classified into groups, and diagnostic texts for the group that the subjects belong to are extracted and displayed. In this instance, the texts are displayed only for the duration of the time limit for the group to which the subject belongs, so the subject can read the texts only during the time limit.

Moreover, the size and quantity of the diagnostic texts are set so that the entire text is displayed on one screen. That is, the texts are set to be displayed within one page on the screen.

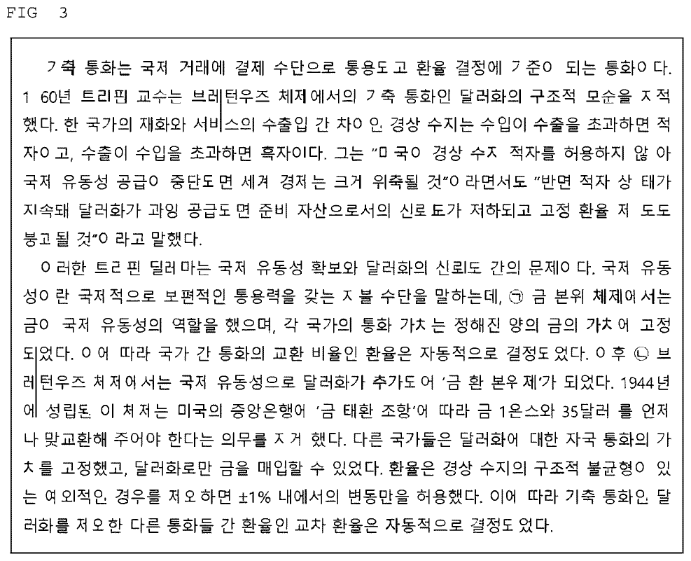

An example of the diagnostic texts displayed on the screen is illustrated in FIG. 3. As illustrated in FIG. 3, the diagnostic text is composed of multiple lines (or rows), and the width of each line (or row) is uniformly set within the total width of the text.

Next, the gaze results on the diagnostic texts tracked by the eye tracking device 20 are acquired (S20).

The eye tracking device 20 tracks the gaze of the subject while the subject is reading the diagnostic texts displayed on the screen and detects the position where the subject is gazing on the texts. The eye tracking device 20 marks the position (area of interest) where the subject's eyes are gazed as a gaze point, and marks larger-sized gaze points as the gaze lingers longer.

Moreover, the subject gazes at one place (area of interest) on the diagnostic text and then moves (jumps) to gaze at another place (area of interest), repeating the process. In this case, the section where the gaze moves from one area of interest to another is a jump section. The areas of interest are marked as gaze points, and the jumps (or skips) between areas of interest are displayed as links between gaze points.

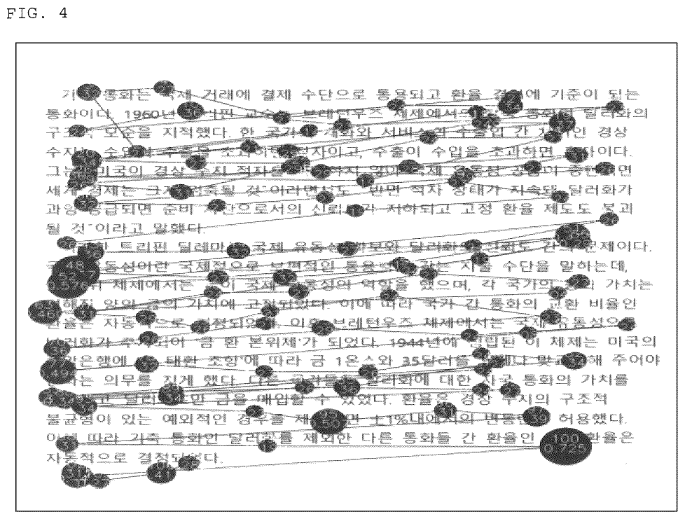

Therefore, as illustrated in FIG. 4, the gaze results obtained by the eye tracking device 20 consist of a plurality of gaze points on the diagnostic text and links connecting the gaze points. The gaze points are displayed as circular or elliptical shapes, and the links connecting the gaze points represent an eye movement or a gaze path. As described above, the size of the gaze point indicates the duration of gaze at the corresponding timepoint. In other words, the size of the gaze point is proportional to the time spent gazing at the gaze point.

Next, the subject's completion of reading is determined from the tracking results (S30).

Particularly, whether the subject has fully read the text is determined by whether the last line of the diagnostic text is marked with gaze points. Preferably, when an area (or reading area) extended by a predetermined ratio around the gaze point is included in the last line (or its area) of the diagnostic text, it is determined that the text has been fully read. Since the gaze points are areas where the subject's eyes are concentrated, it can be assumed that the subject actually reads not only the area of the gaze point but also the surrounding area. Therefore, the actual area read by the subject can be larger than the area of the gaze point.

As described above, the subject can read the diagnostic text only during a set limit time. Therefore, subjects can be primarily classified into those who complete reading the diagnostic text and those who do not.

Next, if the subject does not complete reading, for the subject, it is determined as the Type 1 reading ability deterioration (S40).

The Type 1 reading ability deterioration can occur due to excessive involvement in activities like gaming and video watching. That is, the Type 1 reading ability deterioration represents cases where the subject fails to read the entire diagnostic text, and indicates either slow reading speed (lack of fluency) or insufficient vocabulary.

Preferably, the Type 1 reading ability deterioration can be further diagnosed as either a lack of fluency or vocabulary. In this instance, the Type 1 reading ability deterioration can be diagnosed in detail by the size of the gaze points and the density of the spacing between the gaze points.

More specifically, gaze points larger than a predetermined size (or threshold size) are identified as excessive gaze points. If excessive gaze points are identified, it is diagnosed as a lack of vocabulary. Preferably, if the number of the excessive gaze points exceeds a predetermined number (or threshold number), it is diagnosed as a lack of vocabulary. Furthermore, the threshold size for determining the excessive gaze points can be set to a specific size (for example, 250 ms) in advance or can be set based on past statistics such as the average of past gaze results (particularly, the statistical data of gaze results of normal individuals).

If a subject lacks vocabulary skills, the gaze will fixate on unfamiliar words for a long period of time and the gaze fixation time on such gaze points is lengthened, so the size of the gaze points are enlarged. Thus, a large number of excessive gaze points indicate a deficiency in vocabulary skills.

Additionally, it is determined whether a series of intervals between the gaze points are each smaller than a predetermined interval (or minimum threshold interval), and it is considered as insufficient movement (or insufficient movement of gaze). The interval between the gaze points refers to an interval between adjacent gaze points.

Preferably, if intervals between the gaze points smaller than the minimum threshold interval occur consecutively more than a predetermined number of times, the gaze points are considered as insufficient movement. If determined as the insufficient movement, it is diagnosed as a lack of fluency. Furthermore, the minimum threshold interval can be set to a specific distance in advance or can be set based on past statistical data, such as the average of past gaze results (especially the statistical data of normal subjects' gaze results).

If the subject lacks fluency, the subject reads with reading intervals which are frustratingly narrow and dense. Therefore, the gaze points appear closely packed together, resembling a cluster of grapes.

FIG. 4 exemplifies gaze results determined as a type 1 reading ability deterioration.

Next, if the subject fully reads the text, it is determined whether it is type 2 reading ability deterioration (S50). That is, the type 2 reading ability deterioration is determined by an eye leap pattern or excessive eye leaps for line changes.

The number of characters (or words) displayed within the link between the gaze points is calculated to extract the gaze movement (or skipping of words) or the pattern of the gaze movement. In other words, the number of characters in the diagnostic text appearing between the area (or characters of the area) of a certain gaze point and the next gaze point is calculated, and it is referred to as ‘the number of characters in a link.’

The pattern of eye movement is the number of characters in a series of links according to the order of the gaze points. For example, if the number of characters in the first link (between the first and second gaze points) is 3, the number of characters in the second link (between the second and third gaze points) is 5, and the number of characters in the third link is 7, the pattern of eye leaps is 3, 5, 7, etc.

Furthermore, links where the starting point (starting gaze point) and endpoint (ending gaze point) appear on different lines are referred to as ‘line change links.’ That is, the line change link represents a link showing the gaze movement when the subject changes lines while reading the diagnostic text. The number of characters in the line change link means the number from the character at the starting gaze point to the last character of that line, and the characters from the first character of the next line to the character at the ending gaze point of the next line. Additionally, links that are not the line change links, that is, links where the starting point (starting gaze point) and endpoint (ending gaze point) appear on the same line, are referred to as ‘within-line links.’

Furthermore, if the calculated number of characters exceeds a predetermined number (or threshold character count), such eye movement (link of gaze points) is determined as excessive eye leaps (or word skipping). Links (links of gaze points) where excessive eye leaps or word skipping occur are referred to as ‘excessive leap links (overleaped links).’

In the case of Korean language, since the maximum number of characters that can be read at once is seven, skipping over more than seven characters implies that the intervening characters were not fully read. Therefore, after setting the threshold character count to seven characters, if more than seven characters are skipped, such eye movement (or link) is determined as excessive eye leaps or word skipping (excessive leap links).

Alternatively, the threshold character count can be set to the average number of characters typically skipped.

In addition, a different threshold character count can be set for the excessive leaps at line changes (or the number of characters in line change links). Preferably, the threshold character count for excessive leaps at line changes is set to four characters. In the case of Korean language, it is common to skip about one to three characters at the beginning of a line. Moreover, it is also common to shift gaze to the next line before reading the last two to three characters of the line.

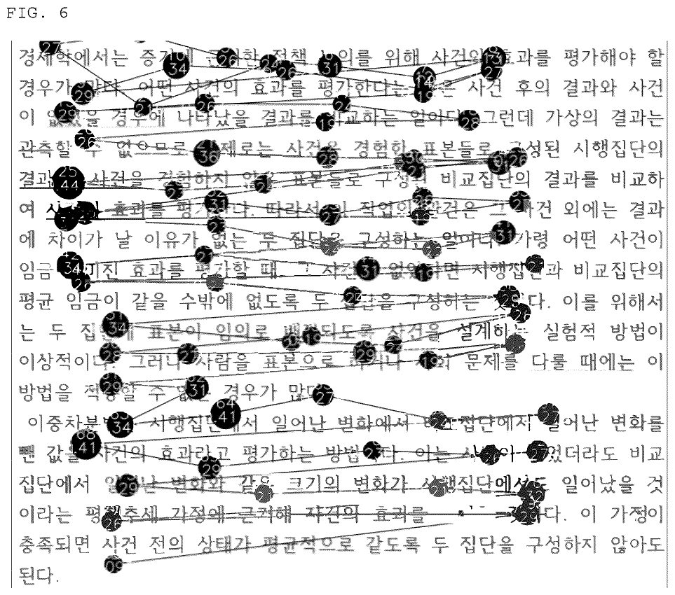

Specifically, line change excessive leaps refer to the excessive leaps that occur between the last gaze point of the previous (current) line and the first gaze point of the next line. FIG. 6 illustrates an example of line change excessive leaps.

Meanwhile, if the number or proportion of line change excessive leaps exceeds a predetermined number or proportion, it is determined as the type 2 reading ability deterioration. In this instance, the proportion of line change excessive leaps represents the ratio of the number of line change excessive leaps to the total number of line changes.

The type 2 reading ability deterioration is known as SNS-type reading ability deterioration, occurring due to a reduction in field of vision caused by excessive SNS usage. That is, the reduction in field of vision leads to consistent line changes or word skipping at the beginning and end of lines.

FIG. 6 illustrates an example determined as the type 2 reading ability deterioration.

Next, if the subject completes reading and does not exhibit the type 2 reading ability deterioration, the subject is considered normal.

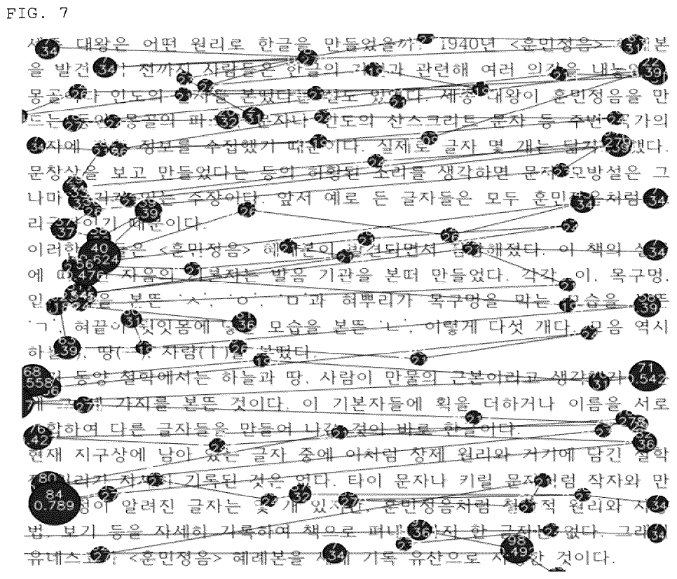

That is, when the subject completes reading, even if within-line excessive eye leaps occur, it is deemed to be normal. For instance, if within-line excessive eye leaps appear uniformly, it is considered as a normal case of constant-speed reading. This case refers to regularly (uniformly) skipping words beyond the average cognitive range of characters. That is, the subject is considered to have normal reading ability, and to possess better-than-average reading ability. Therefore, the subject is not only normal but also exhibits superior reading skills. FIG. 7 illustrates an example of gaze results determined as constant-speed type.

The eye tracking device 20 detects, if the duration of gaze fixation at a point exceeds a threshold time, the point as a gaze point. Therefore, even if the subject actually read, if the gaze fixation time is shorter than the threshold time, the eye tracking device 20 fails to detect a gaze point. In other words, due to the measurement error of the eye tracking device 20 and the size of the threshold time, the eye tracking device 20 cannot detect gaze points accurately, so does not determine within-line excessive eye leaps as reading ability deterioration.

However, in the case of line change excessive leaps, when the gaze moves from the last gaze point on the previous line to the first gaze point on the next line, since the direction of eye movement changes from right to left, the gaze lingers at the last gaze point on the previous line. Therefore, the likelihood of reading the words between the last gaze point on the previous line and the first gaze point on the next line is very low.

While the present invention by the inventor has been described in detail according to an embodiment, it should be understood that the present invention is not limited to the described embodiment and can be variously modified within the scope that does not depart from the gist of the invention.

What is claimed is:

1. A high-speed reading test method using eye tracking, which displays diagnostic texts through a display device, and acquires gaze results of a subject who has read the diagnostic texts by an eye tracking device to assess reading ability, comprising the steps of:
(a) outputting diagnostic texts to be tested, and limiting to read only during a pre-determined test duration;
(b) tracking the gaze of the subject to acquire gaze results within a text area, wherein the gaze results includes gaze points displayed within the diagnostic texts, and links connecting the gaze points;
(c) determining, based on the gaze results of the subject, whether the text has been fully read; and,
(d) classifying the subject's reading ability based on whether the text is fully read or not within the limited time.

2. The high-speed reading test method according to claim 1, wherein in the step (d), when the text has not been not fully read within the limited time, a type 1 reading ability deterioration is determined.

3. The high-speed reading test method according to claim 1, wherein in the step (d), when the text has not been fully read within the limited time, a lack of vocabulary is determined by excessive gaze points larger than a pre-determined threshold size, or a lack of fluency is determined by using insufficient movement, where a series of intervals between the gaze points each move less than a pre-determined minimum threshold interval.

4. The high-speed reading test method according to claim 1, wherein in the step (d), when the text has been fully read within the limited time, a type 2 reading ability deterioration is determined based on whether there are excessive leaps in the eye leaps at line changes of the diagnostic text.

5. The high-speed reading test method according to claim 1, wherein in the step (d), when the text has been fully read within the limited time, if the number of characters in a line change link exceeds a pre-determined threshold character count, it is determined that excessive leaps at line changes (hereinafter, line change excessive leaps) have occurred, and if the number or proportion of line change excessive leaps exceeds a pre-determined number or proportion, it is determined as the type 2 reading ability deterioration.
